(19) United States
(12) Patent Application Publication
(10) Pub. No.: US 2023/0270496 A1
(43) Pub. Date: Aug. 31, 2023

(54) SPECTROSCOPIC SIGNAL DETECTION DURING A LASER PROCEDURE

(71) Applicant: GYRUS ACMI, INC. D/B/A OLYMPUS SURGICAL TECHNOLOGIES AMERICA, Westborough, MA (US)

(72) Inventors: Kurt G. Shelton, Bedford, MA (US); Sergey A. Bukesov, Acton, MA (US)

(21) Appl. No.: 18/166,275

(22) Filed: Feb. 8, 2023

Related U.S. Application Data
(60) Provisional application No. 63/268,525, filed on Feb. 25, 2022.
(60) Provisional application No. 63/371,416, filed on Aug. 15, 2022.

(51) Int. Cl. A61B 18/22

(52) U.S. Cl. CPC A61B 18/22; A61B 2018/00904

(57) ABSTRACT

A system for spectroscopic signal detection can comprise a spectrometer, a light source emitter configurable to emit a first signal toward a target, and an aiming light source emitter configurable to emit a second signal having a visible spectrum toward the target. The system can include a first optical component such as a filter for attenuating or removing noise associated with the second signal from a third signal from the target back to the spectrometer.

### CROSS-REFERENCE TO RELATED APPLICATIONS

This application claims the benefit of priority to U.S. Provisional Patent Application No. 63/268,525 filed Feb. 25, 2022, and U.S. Provisional Patent Application No. 63/371,416 filed Aug. 15, 2022, the contents of which are incorporated herein by reference.

### TECHNICAL FIELD

The present disclosure relates to spectroscopic signal detection during diagnostic or therapeutic laser procedures.

### BACKGROUND

Therapeutic laser systems are used during surgical laser procedures, such as laser lithotripsy, in which a physician may be required to interact with targets such as a tumor or a calculus (“stone”), in a patient's body. Such systems may employ visible light emission, which can function as an aiming beam to provide spatial information of the therapeutic laser either before or while the therapeutic laser light is emitted. Light reflected or otherwise returned from a target may be analyzed using spectroscopy, such as to detect and characterize the target, to differentiate the target from tissue, or the like.

### SUMMARY

A system for spectroscopic signal detection can comprise a spectrometer, a light source emitter configurable to emit a first signal toward a target, and an aiming light source emitter configurable to emit a second signal toward the target. The second signal can be an aiming beam from a source such as a laser diode or a light emitting diode (LED) that can illuminate an area in which an ablation laser may be directed. The first signal can be a signal from a laser source, such as a therapeutic (e.g., an ablation) laser that can be used during a medical procedure to ablate tissue, reduce a stone, or the like. The system can include one or more optical components used with a laser fiber such as a filter, a polarizer, a coated lens, or any similar components for attenuating or removing noise associated with the first signal and/or the second signal from a third signal, such as a scattered signal, a reflected signal, or the like, from the target back to the spectrometer.

The spectrometer can be coupled to the laser fiber and/or to a surgical fiber, which can in turn be coupled to, included in, or a part of an endoscope for performing patient diagnosis or treatment. The one or more optical components can be located in one or more signal pathways, such as a first signal pathway between the surgical fiber and the spectrometer, a second signal pathway between the aiming light source emitter and the surgical fiber and/or an optical path between the target and the spectrometer. An optical coupler or other housing or coating coupled to the laser fiber can be formed from a material capable of absorbing light reflected from a reflector or one or more other optical components that can be included in the housing and used with the laser fiber. In this manner, the light-absorbent housing can help to reduce the amount of stray light directed toward the spectrometer. The system can further include a controller coupled to the light source emitter, the aiming light source emitter, and/or the spectrometer. The controller can be configurable to pulse the aiming light source on and off and cause the spectrometer to collect the signal from the target when the aiming light source emitter is pulsed off.

The controller can also cause the spectrometer to analyze the signal from the target at some time or interval, such as when the aiming light source emitter is pulsed off, or at any time desired whether the aiming is on or off. Hence, the controller can be configured to control the operation or functioning of any light sources and/or the spectrometer included in the system. Thus, the system can improve signal detection such as for spectroscopy analysis, by helping to reduce, lower, or eliminate the influence, interference, or noise resulting from the aiming beam, which can significantly improve the quality of the signals detected from the target.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example of locations for placement of optical components within a laser system.

FIG. 2 illustrates an example of portions of a laser system.

FIG. 3 illustrates an example of a graph of an aiming beam intensity versus time in a blinking mode.

FIG. 4 illustrates an example diagram of a method for spectroscopic signal detection during an in-vivo-insertable medical procedure.

FIG. 5 is a block diagram illustrating an example of a machine upon which one or more embodiments may be implemented.

FIG. 6 illustrates an example of a schematic diagram of an exemplary computer-based clinical decision support system (CDSS).

FIG. 7 illustrates an example of a graph of the effect of the optical components on the visible feedback spectra.

### DETAILED DESCRIPTION

The present systems and methods can help improve spectroscopic signal detection during a diagnostic or therapeutic laser procedure. Surgical laser procedures, such as laser lithotripsy, can use a surgical laser system. The surgical laser system can include a visible light laser and a working laser. The working laser can include a diagnostic or therapeutic laser. The visible light laser can act as an aiming beam, such as to show where the therapeutic or other working laser will be aimed. The visible light laser can also help to provide spatial information (e.g., beam diameter). The beam diameter or other spatial information can be viewed by a physician or other operator, such as through viewing optics of an endoscope or other scope or similar device. Such spatial information can be used (or signal-processed for use) for helping guide or otherwise operate the therapeutic laser either before the therapeutic laser is engaged to emit its laser energy and/or concurrently with the emission of energy by the therapeutic laser. Such a laser system can also include a response light detector, such as a spectrometer to provide spectral analysis of a target (or a material component of a target) such as within a body of a patient. The spectrometer can be used to detect and characterize the target or to differentiate the target from healthy tissue (e.g., to determine the margins of a tumor). For example, light (visible or non-visible) reflected, scattered, or otherwise emitted from the target toward a detector included in, within, or coupled to the laser system can be collected by the spectrometer, such as for signal-processing and spectroscopic analysis. The spectrometer can analyze signals originating from the target and, based thereon, can determine a characteristic associated with the target. The characteristic of the target can include, for example, the composition of the target, hardness, density, or any similar characteristic. In addition, one or more of the laser settings (e.g., laser intensity) can be adjusted based at least in part on the target characteristic.

A “clean” spectroscopy signal can involve:
1. An endoscopic light source to be “on”. The light source can include a xenon light source. The light source can include a continuous, non-pulsed emission lamp or a LED light source. The LED light source can be pulsed, such as to achieve lower average intensities (as determined by the duty cycle of the pulsing). When the LED light source is being pulsed “on”, it can emit high-intensity optical energy. When the LED light source is not being pulsed “on”, it emits no optical energy, and as a result will have no spectra.
2. Laser emission to be “off”. This can reduce or eliminate the occurrence of high-intensity emissions attributable to thermal degradation effects. The high-intensity emission can take time (e.g., approximately 1 millisecond) to decay. Therefore, spectral data can be collected a short time (1 ms) after a laser emission pulse ends.
3. The aiming beam to be “off” or at a low enough intensity that the optical filters effectively reduce or minimize any distortion to the spectral profile due to the aiming beam. Effects of a blinking aiming beam can be avoided in the measured spectra, such as by collecting the spectra between aiming beam pulses.

The aiming beam can impose challenges for signal detection over a range of wavelengths (spectrum spread) of interest in spectrometer analysis. This is because the aiming beam can have a larger light intensity than response signals of interest that can be reflected, scattered, or otherwise emitted from a target object. This can cause an undesirable signal-to-noise ratio (SNR) in the return or response signal from the target when the aiming beam is being emitted. For example, the signal from the aiming beam can potentially “swamp out”, interfere with, or override the response signal of interest from the target, particularly in cases in which the response signal from the target falls within the visible spectrum, such as similar to the aiming beam. The present disclosure provides examples of an approach to improve signal detection such as for spectroscopy analysis, by helping to reduce, lower, or eliminate the influence, interference, or “noise” resulting from the aiming beam, which can significantly improve the quality of the signals detected from the target.

A system for spectroscopic signal detection can comprise a spectrometer, a light source emitter configurable to emit a first signal toward a target, an aiming light source emitter configurable to emit a second signal having a visible spectrum toward the target, and a first optical component for attenuating or removing noise associated with the second signal from a third signal from the target back to the spectrometer. The third signal from the target can be a reflected signal, a scattered signal (e.g., via RAMAN scattering), fluorescence, or the like.

In an example, the spectrometer can be coupled to a surgical fiber, such as attached to the surgical fiber or communicatively coupled to the surgical fiber, and the first optical component can be located in a first signal pathway between the surgical fiber and the spectrometer. The first optical component can be a filter or a polarizer and can be located in any portion of the first signal pathway, such as at an interface between the surgical fiber and the spectrometer, or any other desired location. Alternatively, the first optical component can be located in a second signal pathway between the aiming light source emitter and the surgical fiber or the spectrometer, or in an optical path between the target and the spectrometer.

The system can additionally include a second optical component for attenuating or removing noise associated with at least one of the second signal or the third signal. The second optical component can be located in the first signal pathway or the second signal pathway, and the second optical component can include a filter or a polarizer. Hence, the system can include one or more optical components, such as a filter or a polarizer. The optical component(s) can be located in a signal pathway between the surgical fiber and the spectrometer, between the aiming light source emitter and the surgical fiber or spectrometer, and/or in an optical path between the target and the spectrometer. The system can include as many optical components as desired located in any signal path or optical path desired for attenuating or removing noise associated with the second signal.

The system can further include a controller coupled to the light source emitter, the aiming light source emitter and/or the spectrometer. The controller can be configurable to pulse the aiming light source on and off, and the spectrometer can be configured to collect or analyze at least one of the first signal or the third signal when the aiming light source is pulsed off. Additionally, or alternatively, the spectrometer can be configured to collect or analyze the first signal and/or the third signal when the second signal (i.e., the aiming light source) is pulsed off. The controller can further be configured to control the operation of the light source emitter and/or the spectrometer.

The controller or the spectrometer can further be configured to pulse the aiming light source to allow for a change in a wavelength (a first wavelength) of the light emitted from the aiming light source to a different wavelength (a second wavelength) to occur or happen. When the aiming light source is being emitted in the second wavelength, the spectrometer can analyze the first signal and/or the third signal from the target in response to the second signal at the second wavelength. For example, the aiming light source can be changed or pulsed from a wavelength in the visible spectrum to a wavelength in the non-visible spectrum, and the spectrometer can analyze the first signal and/or the third signal when the light source is emitting light in the non-visible spectrum. It should be understood that throughout this disclosure, the term light source and light source emitter can be used interchangeably. Similarly, the terms aiming light source, aiming beam emission source, and aiming light source emitter can be used interchangeably. Or stated another way, the term light source and light source emitter are understood to refer to the same component, and the term aiming light source, aiming beam emission source, and aiming light source emitter are understood to refer to the same component, regardless of whether the word emitter is included.

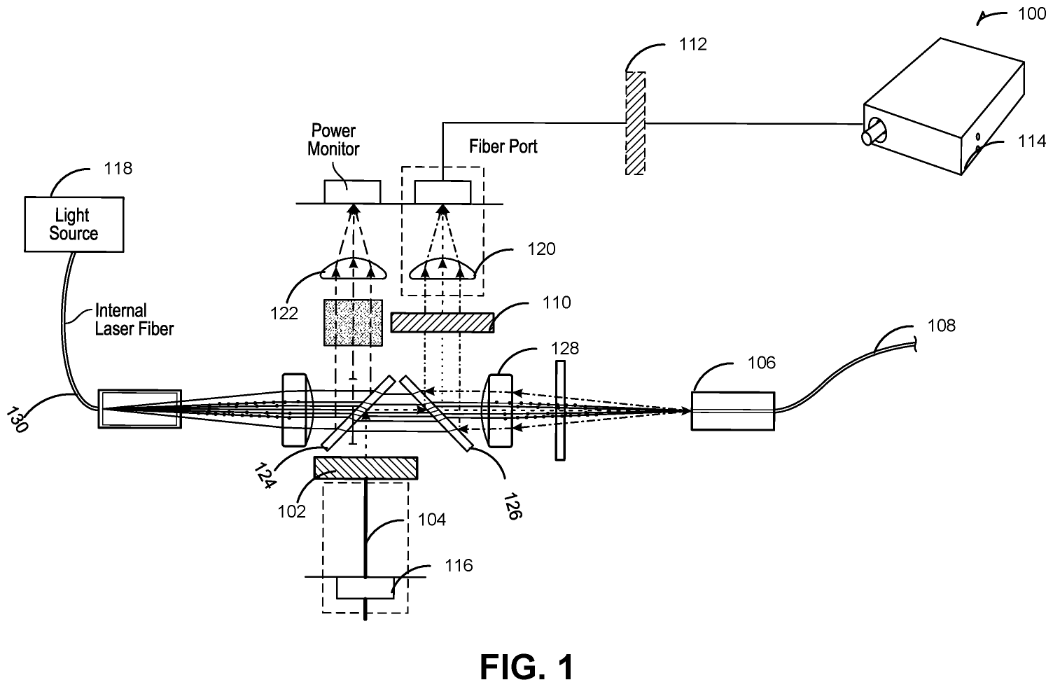

FIG. 1 illustrates an example of locations for placement of one or more optical components within a laser system 100. The laser system 100 can be coupled to an endoscopic system, such as an in-vivo insertable therapeutic or diagnostic endoscopic system, for performing patient diagnosis or treatment. Details regarding how the laser system 100 can be connected to an endoscopic system can be found in U.S. patent application Ser. No. 16/984,447, the contents of which are incorporated in their entirety. In the example illustrated in FIG. 1, a first optical component 102 such as a laser filter or a polarizer can be located at the output of an aiming beam emission source (e.g., a laser diode) 116 in a signal pathway of the aiming beam 104. The system can also include a light source 118 (e.g., a laser module or component), which can emit a signal such as laser radiation to ablate tissue, break up a stone (e.g., a kidney stone or a gallstone) or perform any suitable therapeutic or diagnostic procedure, and can emit signals in the visible spectrum or the non-visible spectrum. The first optical component 102 can be at least equal to or slightly larger than the diameter of the aiming beam 104. The first optical component 102 can remove the sources of noise (e.g., the spectrum spread of the main frequency/wavelength of the aiming beam 104), which can significantly improve the signals detected from the target 106.

Thus, in the example of FIG. 1, a signal emitted from the aiming beam emission source 116 can be filtered, attenuated, blocked, polarized, or otherwise affected by the first optical component 102, so that only signals of desired wavelengths and intensities are emitted from the surgical fiber 108 and reach the target 106. In an example, at least a portion of a signal emitted from the surgical fiber 108 can be reflected back from the target 106 (as denoted by the arrows). The laser system 100 can additionally include one or more additional optical components (e.g., second optical component(s)), such as one or more notch filters 110 and 112 (or any suitable filter(s)) located in an optical path between the target 106 and the spectrometer located in the feedback box 114. The notch filters 110 and 112 can be used to remove any reflection signals reflected back from the target 106 with frequencies or wavelengths around, near, or substantially close to those of the aiming beam 104.

In an example, the width of high attenuation wavelengths of the notch filters 110 and 112 (or any suitable filter(s)) can be greater than the Full Width at Half Maximum (FWHM) specification of the aiming beam emission source 116 since the intensity of the aiming beam 104 can be orders of magnitude greater than the signal from the target (e.g., a spectroscopic signal). Therefore, even an apparently steep wavelength rise provided by the first optical component 102, or directly from the aiming beam emission source 116 can have an unacceptable level of interference at the tails of the emission pulse, or at the first optical component 102.

Additionally, or alternatively, one or more third optical components 120, 122 (e.g., a lens) located at or near the output of the aiming beam 104 as it passes through the first optical component 102 (not shown) and/or the optical path between the target 106 and the spectrometer in the feedback box 114 can be coated with a suitable material to provide, replace, or enhance the filtering effects provided by the first optical component 102 and/or the notch filters 110, 112.

Furthermore, as discussed above, any of the first optical component 102, the second optical component 120, and/or the third optical component 122 can be a polarizer that can be used to improve the spectroscopic analysis. In another example, polarizers can be used in conjunction with, as a replacement for, the other optical components such as filters. In an example, one or more additional optical components 124, 126 can be used to aid or assist with directing the aiming beam 104. For example, first additional optical component 124, can be located in front of the aiming beam emission source 116, which can be a laser diode, or a light emitting diode (LED), and aid in directing the aiming beam 104 in a first direction (e.g., horizontally or in the “x” direction). Similarly, a second additional optical component 126 can be located near a VIS lens 128 (or any lens optimized to operate in the visible spectrum of light, or a range of 400-700 nanometers (nm)) and direct beams in a direction substantially perpendicular to the first direction (e.g., vertically or in the “y” direction). The second additional optical component 126 can thus block the aiming beam. Therefore, the optical feedback signal from the target can be transmitted through a VIS optical port to the spectrometer in the feedback box 114.

The laser radiation emitted from the light source 118 may have the same or different wavelength or frequency from that of the aiming beam 104 emitted from the aiming beam emission source 116. It is understood that any of the optical components discussed above can be used to block, attenuate, redirect, or the like, a portion of the laser radiation associated with the aiming beam 104 that is either emitted from the aiming beam emission source 116 or returned from the target 106 during a medical procedure.

The frequency associated with the specification of any of the optical components, such as the first optical component 102, the notch filters 110, 112, or the like, can be dependent on the wavelength of the aiming beam 104. The wavelength of the aiming beam 104 may drift or vary by an amount, such as 1-2 nanometers due to external factors (e.g., temperature) and thus the one or more optical components can be selected to account for such a drift. For example, one or more of the optical components can be selected that have a wavelength range or spread, such as a 10 nm range, to account for the wavelength drift of the aiming beam 104. Additionally, or alternatively, an optical component such as a laser filter can be selected to be optimized for the specific aiming beam emission source 116 (e.g., aiming beam laser diode) being used. For example, a rejection frequency of one or more of the optical components can be based on a wavelength of the aiming beam emission source 116 so as to reject frequencies that correspond to that aiming beam wavelength. The various optical components and filters discussed above can be included within a light impenetrable housing that can be coupled to an internal laser fiber 130, which can in turn be coupled to the light source 118, the surgical fiber 108, and/or the feedback box 114.

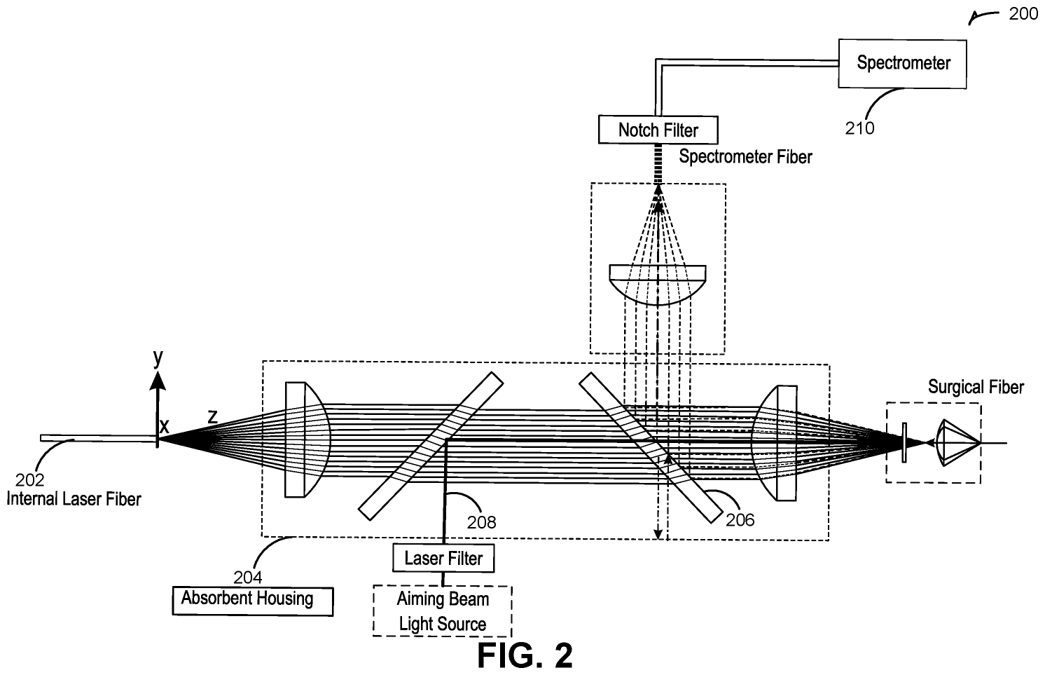

FIG. 2 illustrates an example of portions of a laser system 200. The laser system 200 can include a laser fiber 202 that can be housed or carried in a light-absorbent housing. FIG. 2 illustrates a system 200 including optical components that can be the same as or similar to those illustrated and discussed above with respect to FIG. 1. In FIG. 2, the laser fiber 202 can be coupled to a housing 204, such as which can house certain of the optical components. The housing 204 can be configured to absorb light reflected off of an optical component housed therein, such as a reflector 206. For example, the housing 204 coupled to the internal laser fiber 202 can be formed from or coated with a material that can substantially (e.g., 90% or, in some embodiments, 80%) absorb (e.g., not reflect) light of a specific wavelength or range of wavelengths within which the light from an aiming beam 208 is included. In an example, the reflector 206 can perform the same or similar function as the second additional optical component 126 as described for FIG. 1, specifically to direct light from the aiming beam 208. The reflector 206 can direct light in a direction toward the housing 204 (as denoted by the arrow pointing from the aiming beam 208 toward the housing 204). Thus, light from the aiming beam 208 (and/or reflections from the target) and directed by the reflector 206 toward the housing 204 can be absorbed by the housing 204 in this embodiment. This can effectively cause the stray light of the aiming beam 208 and/or the reflections from the target to be at least partially blocked and reduce or lower its amount before reaching the spectrometer 210.

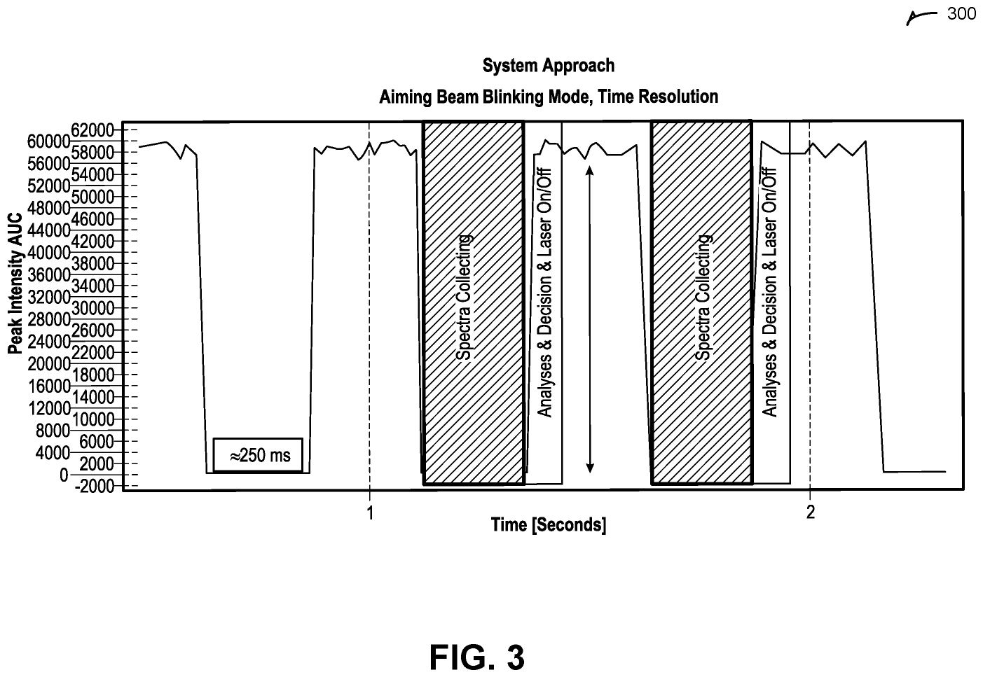

FIG. 3 illustrates an example of a graph 300 of an aiming beam intensity versus time when in a blinking mode. As illustrated in the example of FIG. 3, aiming beam pulses can be modulated to mitigate or reduce its effect on the signal returned from the target (e.g., a spectroscopic signal or other signal of interest to be analyzed by the spectrometer). In an example, the laser diode of the aiming beam can be switched ON and OFF very fast, and the spectrometer may collect data while the aiming beam is switched OFF and check the data or perform analysis on the data when the beam is switched back ON. For example, the aiming beam can be switched OFF for a period of time such as approximately 250 ms, during which time the spectrographic data can be collected. The aiming beam can be switched back ON for another period of time, such as 0.5 seconds to allow the spectrometer to check and analyze the data. These steps can be iteratively performed until a sufficient amount of data is collected for analyzing a characteristic of the target or the medical procedure is complete. The aiming beam can be pulsed periodically, recurrently, or at any desired interval. The interval can be determined and controlled by a controller, or any similar component connected or coupled to the aiming beam emission source. While FIG. 3 illustrates an example of the aiming beam being pulsed OFF for 250 ms, the pulses can occur at a faster rate such as 10-20 ms, or a slower rate, as desired or appropriate for the medical procedure being performed. In an example, the spectra collection time may be shortened or otherwise adjusted when residual optical effects from the aiming beam or laser emission pulses are present. For example, spectroscopic emissions resulting from thermal degradation may take time to decay after the end of the pulse. To avoid capturing that phenomena in the spectra, spectral response collection may be triggered after a short delay after the end of the pulse. Also, while the spectra can be collected between pulses of the aiming beam, such spectra can also be collected during a pulse from any source (e.g., during laser emission pulses, LED source pulses, or the like).

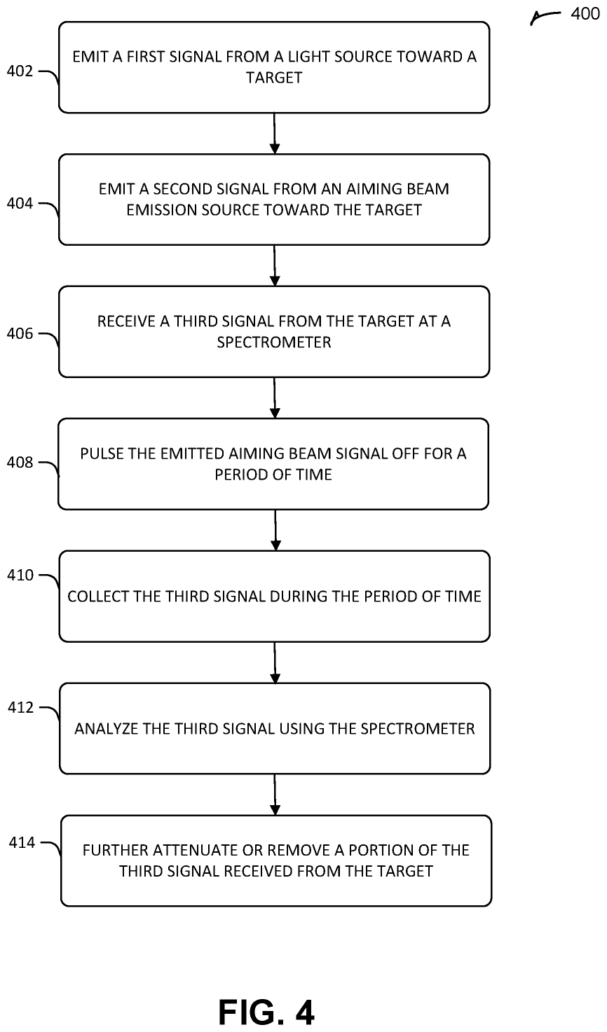

FIG. 4 illustrates an example diagram of a method 400 for spectroscopic signal detection during an in-vivo-insertable medical procedure. The method 400 may include or comprise a number of Operations or Steps (402-412). These Operations are exemplary, and the executed method can omit one or more of the listed Operations, can repeat Operations, can include other Operations, or can execute the Operations concurrently, substantially simultaneously, or in any order, as appropriate or desired.

At 402, a first signal can be emitted from a light source toward a target. The target may be an object such as a kidney stone or a gallstone, a tumor, a piece of tissue or any similar target located within the body of a patient during an in-vivo-insertable medical procedure. The light source can be a laser light, such as emitted from a laser diode, which can be a therapeutic laser (e.g., an ablation laser) used during the medical procedure. The light source can be an endoscopic light source, such as an LED, and as discussed above, the laser system can be coupled to, attached to, or connected to the endoscope.

At 404, a second signal can be emitted from an aiming beam emission source toward the target. The aiming beam can be a visible light emission from a visible light emission source such as a laser diode or an LED that can provide spatial information of the therapeutic laser while or before it is emitted. The spatial information can include a diameter of the therapeutic laser, a radius of the therapeutic laser, a cross-sectional area of the therapeutic laser, a location of the therapeutic laser or any desired or suitable dimension and property of the therapeutic laser.

At 406, a third signal can be received from the target at a spectrometer. The third signal from the target can be a reflected signal, a scattered signal (e.g., via RAMAN scattering), Fluorescence, or the like. For example, at least a portion of the first signal emitted at 402 and/or the second signal emitted at 404 can be reflected back from the target and pass through one or more optical components such as those described in FIG. 1 and collected by the spectrometer.

At 408, the emitted aiming beam signal can be pulsed off for a period of time. A controller or a processor coupled to or connected to the aiming beam emission source may turn the aiming beam signal off for a period of time (e.g., 10 ms, 20 ms, or any appropriate period of time) which can represent a window of time.

At 410, the third signal can be collected when the aiming beam is pulsed off and at 412, can be analyzed using the spectrometer. In an example, during the window of time created when the aiming beam signal is turned off at 408, noise or other influence from the second signal may be eliminated, abrogated, or reduced during the analysis performed at 410. The third signal can represent a spectroscopic signal and the spectroscopic signal may not encompass any signals associated with the aiming beam or at least a significantly attenuated signal associated with the aiming beam, which can allow the third signal to be collected without interference from the second signal. The signal can be analyzed at 412 substantially simultaneously with the collection at 410 (during the period of time the aiming beam if off) or can be analyzed when the aiming beam is back on. Or stated another way, the analysis of the third signal can be performed by the spectrometer regardless of whether the aiming beam is currently on or off.

At 414, at least a portion of the third signal received from the target can be further attenuated or removed. For example, software such as noise reduction software, can be used to remove or attenuate a portion of the third signal that corresponds to, represents, or has the same wavelength as the first signal and/or the second signal (or any other signal that may interfere with the collection of the third signal). This can help ensure that the third signal contains information specific to the target and not from the light source, the aiming beam and/or any other signal (e.g., noise) that may interfere with the collection and/or analysis of the third signal.

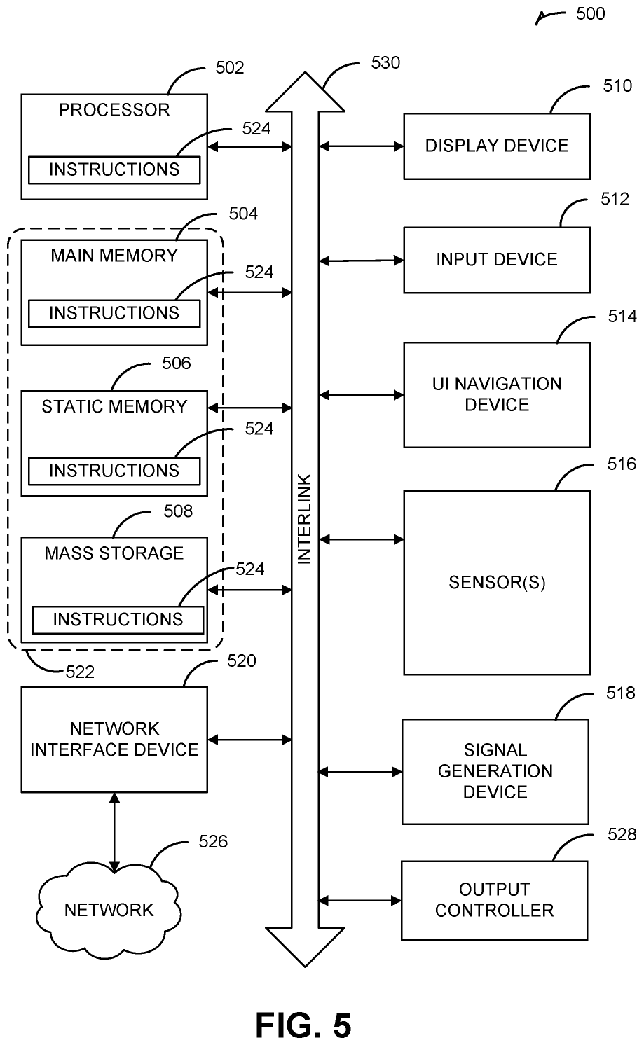

FIG. 5 is a block diagram of an example of a machine 500 upon which any one or more of the techniques (e.g., methodologies) discussed herein may perform. In some embodiments, the machine 500 may operate as a standalone device or may be connected (e.g., networked) to other machines. For example, the machine 500 can be coupled to or connected to the controller and/or the spectrometer to cause the controller or the spectrometer to perform one or more of their operations described above. In a networked deployment, the machine 500 may operate in the capacity of a server machine, a client machine, or both in server-client network environments. In an example, the machine 500 may act as a peer machine in peer-to-peer (P2P) (or other distributed) network environment. The machine 500 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 500 may include a hardware processor 502 (e.g., a central processing unit (CPU), a graphics processing unit (GPU), a hardware processor core, field programmable gate array (FPGA), or any combination thereof), a main memory 504 and a static memory 506, some or all of which may communicate with each other via an interlink (e.g., bus) 530. The machine 500 may further include a display unit 510, an alphanumeric input device 512 (e.g., a keyboard), and a user interface (UI) navigation device 514 (e.g., a mouse). In an example, the display unit 510, input device 512 and UI navigation device 514 may be a touch screen display. The machine 500 may additionally include a storage device (e.g., drive unit) 508, a signal generation device 518 (e.g., a speaker), a network interface device 520, and one or more sensors 516, such as a global positioning system (GPS) sensor, compass, accelerometer, or other sensor. The machine 500 may include an output controller 528, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 508 may include a machine readable medium 522 on which is stored one or more sets of data structures or instructions 524 (e.g., software) embodying or used by any one or more of the techniques or functions described herein. The instructions 524 may also reside, completely or at least partially, within the main memory 504, within static memory 506, or within the hardware processor 502 during execution thereof by the machine 500. In an example, one or any combination of the hardware processor 502, the main memory 504, the static memory 506, or the storage device 516 may constitute machine readable media.

While the machine readable medium 522 is illustrated as a single medium, the term “machine readable medium” may include a single medium or multiple media (e.g., a centralized or distributed database, and/or associated caches and servers) configured to store the one or more instructions 524.

The term “machine readable medium” may include any medium that is capable of storing, encoding, or carrying instructions for execution by the machine 500 and that cause the machine 500 to perform any one or more of the techniques of the present disclosure, or that is capable of storing, encoding, or carrying data structures used by or associated with such instructions. Non-limiting machine readable medium examples may include solid-state memories, and optical and magnetic media. In an example, a massed machine readable medium comprises a machine readable medium with a plurality of particles having invariant (e.g., rest) mass. Accordingly, massed machine-readable media are not transitory propagating signals. Specific examples of massed machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 524 may further be transmitted or received over a communications network 526 using a transmission medium via the network interface device 520 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards), IEEE 802.15.4 family of standards, peer-to-peer (P2P) networks, among others. In an example, the network interface device 520 may include one or more physical jacks (e.g., Ethernet, coaxial, or phone jacks) or one or more antennas to connect to the communications network 526. In an example, the network interface device 520 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 500, and includes digital or analog communications signals or other intangible medium to facilitate communication of such software.

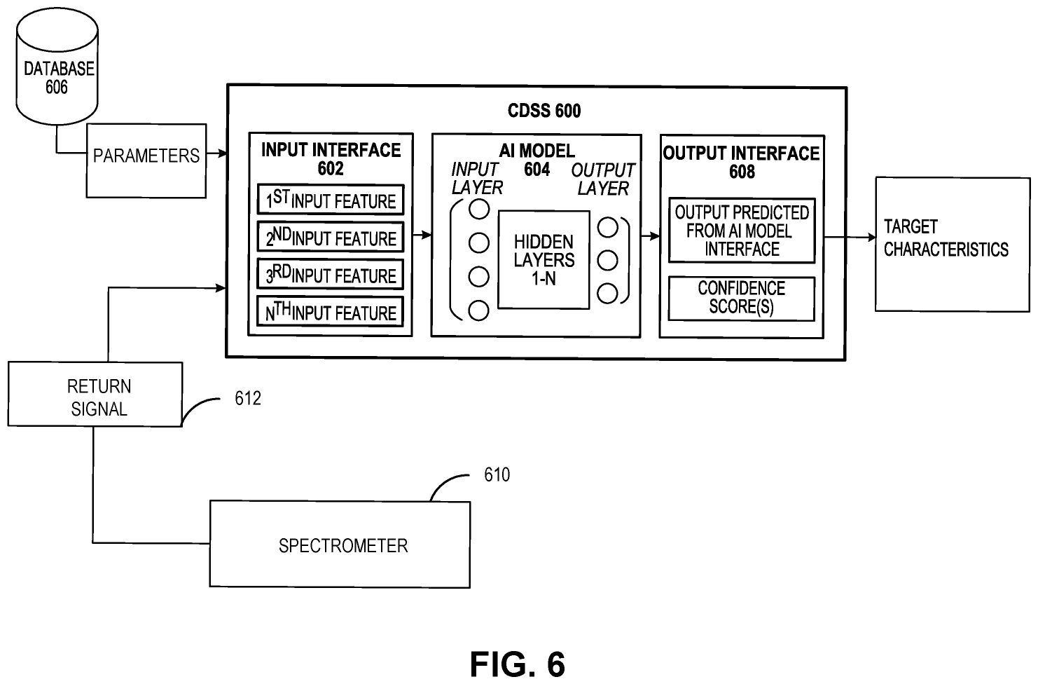

FIG. 6 illustrates a schematic diagram of an exemplary computer-based clinical decision support system (CDSS) 600 that is configured to determine information or characteristics about a target, such as size, composition, hardness, density, or any similar characteristic or information about the target based on spectroscopic analysis of a signal from the target. The CDSS 600 can include an input interface 602 through which parameters such as the size of a surgical fiber, information about the light sources, information about the optical components, and/or information about the scope which are specific to a patient's procedure are provided as input features to an artificial intelligence (AI) model 604, a processor which performs an inference operation in which the parameters are applied to the AI model to generate the determination of the target characteristics, and an output interface 608 through which the determined target characteristics can be communicated to a user, e.g., a clinician.

The input interface 602 may include a direct data link between the CDSS 600 and one or more medical devices that generate at least some of the input features. For example, the input interface 602 may transmit information about the light sources and/or the optical components (e.g., frequency or wavelength of signals from the light sources or aiming beam emission sources, or a rejection frequency of the optical components), or information about a signal returned from the target directly to the CDSS 600 during a therapeutic and/or diagnostic medical procedure. In an example, information about the light sources and/or the optical components, the scope, etc., used during the procedure can be stored in a database 606. Additionally, or alternatively, the input interface 602 may be a classical user interface that facilitates interaction between a user and the CDSS 600. For example, the input interface 604 may facilitate a user interface through which the user may manually enter the information about the surgical fiber, the scope, the optical components, signals to block or allow, etc. Additionally, or alternatively, the input interface 602 may provide the CDSS 600 with access to an electronic patient record or the components being used during the procedure from which one or more input features may be extracted. In any of these cases, the input interface 602 is configured to collect one or more of the following input features in association with one or more of a specific patient, a type of medical procedure, a type of scope, signals that should be rejected during spectroscopic analysis, or the like, on or before a time at which the CDSS 600 is used to assess the input features will take place.

An example of an input feature can include a dimension of the surgical fiber to be used during the procedure.

An example of an input feature can include a type of light or laser source.

An example of an input feature can include the type of scope being used during the procedure.

An example of an input feature can include a wavelength or frequency to be blocked or attenuated.

An example of an input feature can include an amount of time to turn a light or laser source off.

An example of an input feature can include signal information of a return signal 612 received at the spectrometer 610 from the target.

Based on one or more of the above input features, the processor performs an inference operation using the AI model 604 to generate determined characteristics of the target such as the size of the target, the composition of the target, hardness, density, or any similar characteristic. For example, input interface 602 may deliver the one or more of the input features listed above into an input layer of the AI model 604 which propagates these input features through the AI model 604 to an output layer. The AI model 604 can provide a computer system the ability to perform tasks, without explicitly being programmed, by making inferences based on patterns found in the analysis of data. The AI model 604 explores the study and construction of algorithms (e.g., machine-learning algorithms) that may learn from existing data and make predictions about new data. Such algorithms operate by building an AI model from example training data in order to make data-driven predictions or decisions expressed as outputs or assessments.

Examples of two modes for machine learning (ML) can include: supervised ML and unsupervised ML. Supervised ML uses prior knowledge (e.g., examples that correlate inputs to outputs or outcomes) to learn the relationships between the inputs and the outputs. The goal of supervised ML is to learn a function that, given some training data, best approximates the relationship between the training inputs and outputs so that the ML model can implement the same relationships when given inputs to generate the corresponding outputs. Unsupervised ML is the training of an ML algorithm using information that is neither classified nor labeled and allowing the algorithm to act on that information without guidance. Unsupervised ML is useful in exploratory analysis because it can automatically identify structure in data.

Tasks for supervised ML can include classification problems and regression problems. Classification problems, also referred to as categorization problems, aim at classifying items into one of several category values (for example, is this object an apple or an orange?). Regression algorithms aim at quantifying some items (for example, by providing a score to the value of some input). Some examples of supervised-ML algorithms are Logistic Regression (LR), Naive-Bayes, Random Forest (RF), neural networks (NN), deep neural networks (DNN), matrix factorization, and Support Vector Machines (SVM).

Some possible tasks for unsupervised ML include clustering, representation learning, and density estimation. Some examples of unsupervised-ML algorithms are K-means clustering, principal component analysis, and autoencoders.

Another type of ML is federated learning (also known as collaborative learning) that trains an algorithm across multiple decentralized devices holding local data, without exchanging the data. This approach stands in contrast to traditional centralized machine-learning techniques where all the local datasets are uploaded to one server, as well as to more classical decentralized approaches which often assume that local data samples are identically distributed. Federated learning enables multiple actors to build a common, robust machine learning model without sharing data, thus allowing to address critical issues such as data privacy, data security, data access rights and access to heterogeneous data.

In some examples, the AI model 604 may be trained continuously or periodically prior to performance of the inference operation by the processor. Then, during the inference operation, the patient specific input features provided to the AI model 604 may be propagated from an input layer, through one or more hidden layers, and ultimately to an output layer that corresponds to the information about the target. For example, when evaluating the spectroscopic analysis of the signal from the target, the system can determine one or more characteristics of the target.

During and/or subsequent to the inference operation, the information about the target can be communicated to the user via the output interface 608 (e.g., a user interface (UI)) and/or automatically cause a surgical laser connected to the processor to perform a desired action. For example, based on the composition of the target the system may cause the surgical laser to emit energy to ablate the target, adjust the amount of ablation energy, move a portion of the scope, etc.

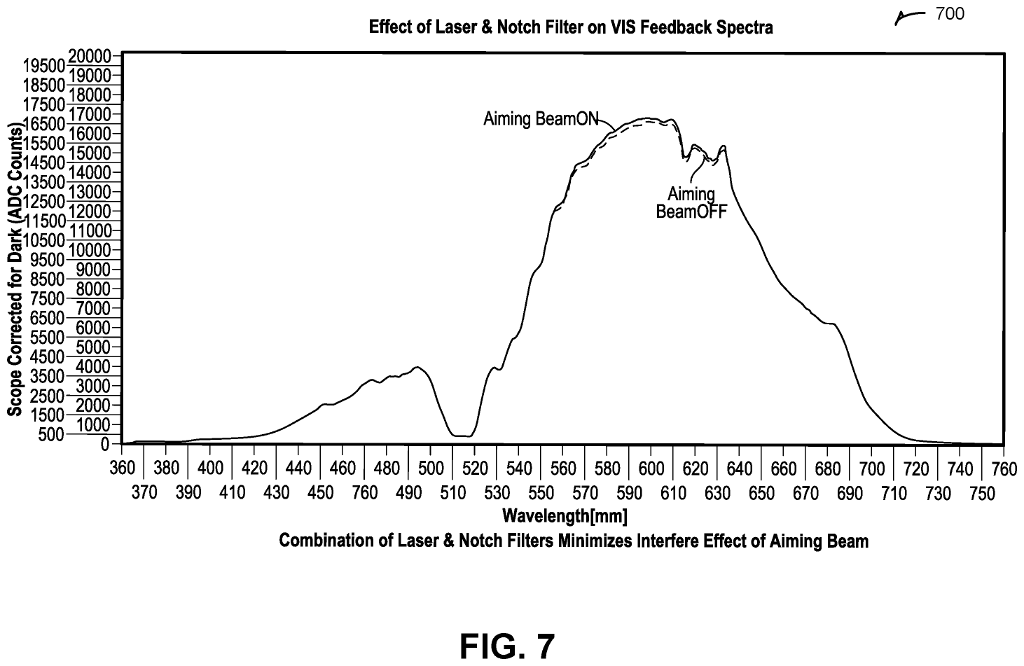

FIG. 7 illustrates an example of a graph 700 of the effect of implementing the first, second and/or third optical component(s) (e.g., a laser and/or a notch filter or any suitable filters, optical components with coatings, and/or polarizers) in the laser system 100 as described above on the visible feedback spectra. As depicted in FIG. 7, when using the laser filter 102 and at least one notch filter 110, 112, improved detection of the spectroscopic signals (e.g., RAMAN, Fluorescence, or Reflectance) can be made substantially even when the aiming beam 104 is actively emitting. FIG. 2 shows a graph of the return signal from the target 106 when the aiming beam is ON (denoted by the solid line) and when the aiming beam is OFF (denoted by the dashed line). Therefore, using a laser filter and/or a notch filter (or any suitable filters, optical components with coatings, and/or polarizers discussed above) results in the curve received when the aiming beam is ON significantly overlapping the curve received when the aiming beam is OFF. Thus, as illustrated in FIG. 7, using the optical components described above can aid to minimize or reduce the interfering effect (e.g., noise) of the aiming beam signal in the signal received back from the target 106.

In an example, the aiming beam 104 can be pulsed, such as pulsed ON and OFF to allow for the spectrometer to collect signals from the target 106 and analyze or measure the collected signals. Stated another way, the aiming beam emission source 116 can be pulsed ON and OFF, so that the aiming beam 104 “blinks”, and the signal from the target 106 can be collected while the aiming beam 104 is OFF and analyzed by the spectrometer when the aiming beam is ON. In such an example, the aiming beam 104 can be pulsed fast enough (e.g., 10-20 millisecond (ms) pulses) that the blinking does not interfere with or distract a surgeon during a medical procedure. In another example, the aiming beam emission source 116 can be pulsed to allow for a change in the wavelength of the aiming beam 104 (e.g., from a first wavelength to a different, second wavelength (e.g., from a visible wavelength to a non-visible wavelength)) so that the aiming beam 104 appears to be continuously ON but is not optically visible while the signal from the target 106 is collected. In such an example, the optical components discussed above may block or attenuate a signal corresponding to the second wavelength.

### ADDITIONAL NOTES AND EXAMPLES

Example 1 is a system for spectroscopic signal detection, the system comprising: a spectrometer; a light source emitter configurable to emit a first signal toward a target; an aiming light source emitter configurable to emit a second signal having a visible spectrum toward the target; and a first optical component for attenuating or removing noise associated with the second signal from a third signal, in response to the first signal, from the target back to the spectrometer.

In Example 2, the subject matter of Example 1 optionally includes wherein the spectrometer is coupled to a surgical fiber, and wherein the first optical component includes at least one of a filter or a polarizer located in a first signal pathway between the surgical fiber and the spectrometer.

In Example 3, the subject matter of Example 2 optionally includes wherein the first optical component includes at least one of a filter or a polarizer located in a second signal pathway between the aiming light source emitter and at least one of the surgical fiber or the spectrometer.

In Example 4, the subject matter of any one or more of Examples 2-3 optionally include a second optical component for attenuating or removing noise associated with at least one of the second signal or the third signal, wherein the second optical component is located in at least one of a first signal pathway between the surgical fiber and the spectrometer or a second signal pathway between the aiming light source emitter and at least one of the surgical fiber or the spectrometer.

In Example 5, the subject matter of Example 4 optionally includes wherein the second optical component includes a filter or a polarizer.

In Example 6, the subject matter of any one or more of Examples 1-5 optionally include wherein the first optical component includes at least one of filter or a polarizer located in an optical path between the target and the spectrometer.

In Example 7, the subject matter of any one or more of Examples 1-6 optionally include wherein the aiming light source emitter includes a laser diode.

In Example 8, the subject matter of any one or more of Examples 1-7 optionally include wherein the light source emitter includes a Light Emitting Diode (LED).

In Example 9, the subject matter of any one or more of Examples 1-8 optionally include wherein a rejection frequency of the first optical component is based on a wavelength of light emitted from the aiming light source emitter.

In Example 10, the subject matter of any one or more of Examples 1-9 optionally include wherein the system is configured to be coupled to an in-vivo-insertable therapeutic or diagnostic endoscopic system.

In Example 11, the subject matter of any one or more of Examples 1-10 optionally include a controller, coupled to the aiming light source emitter, configurable to pulse the aiming light source on and off, wherein the spectrometer is configured to collect or analyze at least one of the first signal or the third signal when the aiming light source is pulsed off.

In Example 12, the subject matter of any one or more of Examples 1-11 optionally include wherein the spectrometer is configured to pulse the aiming light source emitter for allowing a change in a wavelength of light emitted from the aiming light source emitter to a different wavelength.

Example 13 is a system for spectroscopic signal detection, the system comprising: a spectrometer; a light source emitter configurable to emit a first signal toward a target; an aiming light source emitter configurable to emit a second signal having a visible spectrum toward the target; and a controller, coupled to the aiming light source emitter, configurable to pulse the aiming light source emitter on and off, wherein the spectrometer is configured to collect or analyze at least one of the first signal or a third signal, in response to the first signal, from the target when the aiming light source emitter is pulsed off.

In Example 14, the subject matter of Example 13 optionally includes wherein the spectrometer is configured to collect or analyze at least one of the first signal or the third signal when the aiming light source emitter is pulsed on.

In Example 15, the subject matter of any one or more of Examples 13-14 optionally include wherein the spectrometer is configured to pulse the aiming light source emitter for allowing a change in a wavelength of light emitted from the aiming light source emitter to a different wavelength.

Example 16 is a method for spectroscopic signal detection during an in-vivo-insertable medical procedure, the method comprising: emitting a first signal from a light source toward a target; emitting a second signal from an aiming beam emission source toward the target; receiving, in response to the first signal, a third signal from the target at a spectrometer; pulsing the aiming beam emission source off for a period of time; collecting the third signal received during the period of time; and analyzing the collected third signal using the spectrometer.

In Example 17, the subject matter of Example 16 optionally includes attenuating or removing a portion of the third signal received from the target.

In Example 18, the subject matter of Example 17 optionally includes wherein the portion of the third signal is attenuated or removed using at least one optical component in at least one of a first signal pathway between a surgical fiber and the spectrometer or a second signal pathway between the aiming beam emission source and at least one of the surgical fiber or the spectrometer.

Example 19 is a method for spectroscopic signal detection during an in-vivo-insertable medical procedure, the method comprising: emitting a first signal from a light source toward a target; emitting a second signal from an aiming beam emission source toward the target; receiving, in response to the first signal, a third signal from the target at a spectrometer; attenuating or removing noise associated with the second signal from a third signal; and analyzing the collected third signal to determine a characteristic of the target.

In Example 20, the subject matter of Example 19 optionally includes wherein the attenuated or removed noise has substantially the same wavelength as the second signal.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

What is claimed is:

1. A system for spectroscopic signal detection, the system comprising:
a spectrometer;
a light source emitter configurable to emit a first signal toward a target;
an aiming light source emitter configurable to emit a second signal having a visible spectrum toward the target; and
a first optical component for attenuating or removing noise associated with the second signal from a third signal, in response to the first signal, from the target back to the spectrometer.

2. The system of claim 1, wherein the spectrometer is coupled to a surgical fiber, and wherein the first optical component includes at least one of a filter or a polarizer located in a first signal pathway between the surgical fiber and the spectrometer.

3. The system of claim 2, wherein the first optical component includes at least one of a filter or a polarizer located in a second signal pathway between the aiming light source emitter and at least one of the surgical fiber or the spectrometer.

4. The system of claim 2, further comprising:
a second optical component for attenuating or removing noise associated with at least one of the second signal or the third signal, wherein the second optical component is located in at least one of a first signal pathway between the surgical fiber and the spectrometer or a second signal pathway between the aiming light source emitter and at least one of the surgical fiber or the spectrometer.

5. The system of claim 4, wherein the second optical component includes a filter or a polarizer.

6. The system of claim 1, wherein the first optical component includes at least one of filter or a polarizer located in an optical path between the target and the spectrometer.

7. The system of claim 1, wherein the aiming light source emitter includes a laser diode.

8. The system of claim 1, wherein the light source emitter includes a Light Emitting Diode (LED).

9. The system of claim 1, wherein a rejection frequency of the first optical component is based on a wavelength of light emitted from the aiming light source emitter.

10. The system of claim 1, wherein the system is configured to be coupled to an in-vivo-insertable therapeutic or diagnostic endoscopic system.

11. The system of claim 1, further comprising:
a controller, coupled to the aiming light source emitter, configurable to pulse the aiming light source on and off, wherein the spectrometer is configured to collect or analyze at least one of the first signal or the third signal when the aiming light source is pulsed off.

12. The system of claim 1 wherein the spectrometer is configured to pulse the aiming light source emitter for allowing a change in a wavelength of light emitted from the aiming light source emitter to a different wavelength.

13. A system for spectroscopic signal detection, the system comprising:
a spectrometer;
a light source emitter configurable to emit a first signal toward a target;
an aiming light source emitter configurable to emit a second signal having a visible spectrum toward the target; and
a controller, coupled to the aiming light source emitter, configurable to pulse the aiming light source emitter on and off, wherein the spectrometer is configured to collect or analyze at least one of the first signal or a third signal, in response to the first signal, from the target when the aiming light source emitter is pulsed off.

14. The system of claim 13, wherein the spectrometer is configured to collect or analyze at least one of the first signal or the third signal when the aiming light source emitter is pulsed on.

15. The system of claim 13, wherein the spectrometer is configured to pulse the aiming light source emitter for allowing a change in a wavelength of light emitted from the aiming light source emitter to a different wavelength.

16. A method for spectroscopic signal detection during an in-vivo-insertable medical procedure, the method comprising:
emitting a first signal from a light source toward a target;
emitting a second signal from an aiming beam emission source toward the target;
receiving, in response to the first signal, a third signal from the target at a spectrometer;
pulsing the aiming beam emission source off for a period of time;
collecting the third signal received during the period of time; and
analyzing the collected third signal using the spectrometer.

17. The method of claim 16, further comprising:
attenuating or removing a portion of the third signal received from the target.

18. The method of claim 17, wherein the portion of the third signal is attenuated or removed using at least one optical component in at least one of a first signal pathway between a surgical fiber and the spectrometer or a second signal pathway between the aiming beam emission source and at least one of the surgical fiber or the spectrometer.

19. A method for spectroscopic signal detection during an in-vivo-insertable medical procedure, the method comprising:
emitting a first signal from a light source toward a target;
emitting a second signal from an aiming beam emission source toward the target;
receiving, in response to the first signal, a third signal from the target at a spectrometer;
attenuating or removing noise associated with the second signal from a third signal; and
analyzing the collected third signal to determine a characteristic of the target.

20. The method of claim 19, wherein the attenuated or removed noise has substantially the same wavelength as the second signal.
